(19) United States
(12) Patent Application Publication
(10) Pub. No.: US 2022/0282240 A1
(43) Pub. Date: Sep. 8, 2022

(54) METHODS AND DEVICES FOR RARE CELL CAPTURE

(71) Applicants: Sanford Burnham Prebys Medical Discovery Institute, La Jolla, CA (US); Tumorgen, Inc., San Diego, CA (US)

(72) Inventors: Peter TERIETE, San Diego, CA (US); Jeffrey K. ALLEN, La Jolla, CA (US)

(21) Appl. No.: 17/634,533

(22) PCT Filed: Aug. 13, 2020
(86) PCT No.: PCT/US2020/046208

§ 371 Date: Feb. 10, 2022

Related U.S. Application Data
(60) Provisional application No. 62/886,557, filed on Aug. 14, 2019.

(51) Int. Cl. C12N 11/10; B01L 3/00; G01N 33/50

(52) U.S. Cl. CPC C12N 11/10; B01L 3/502761; G01N 33/5091; B01L 2200/0652; B01L 2300/16

(57) ABSTRACT

Disclosed herein is a biomimetic coating for use in a microfluidic channel to capture rare cells from a sample while maintaining the viability of the captured cells.

### CROSS-REFERENCE

This application claims the benefit of U.S. Provisional Application No. 62/886,557, filed Aug. 14, 2019, which application is incorporated herein by reference.

### BACKGROUND OF THE INVENTION

A major cause of cancer-associated mortality is tumor metastasis, the dissemination of tumor cells and tumor cell clusters through the blood circulation system to other tissues. The capture and analysis of these circulating tumor cells and circulating tumor cell clusters from blood samples offers new insights into tumor metastasis and can facilitate the development of cancer treatments. The extremely small numbers of circulating tumor cells require an ability to process a large sample volume rapidly to preserve viable cells. Additionally, the small surface-area to volume ratios of circulating tumor cell clusters reduces the efficiency of capture by an antibody alone. Existing technologies to isolate these cell types are unable to maintain crucial cell viability and cell cluster integrity. New tools are needed to isolate and capture circulating tumor cells as well as circulating metastatic cancer cell clusters. Disclosed herein is a new microfluidic system which can capture rare living individual cells and cell clusters using an easy to functionalize biomimetic hydrogel coating, mimicking the early adhesion events in leukocyte extravasation from human capillary vessels. The present invention allows enhanced capture of rare circulating tumor cells and clusters from fast flowing samples, such as whole blood.

### SUMMARY OF THE INVENTION

Disclosed herein is a biomimetic coating to capture rare cells or rare cell clusters comprising a plurality of cell adhesion molecules specific to a first cell surface feature, a plurality of cell capture molecules specific to a second cell surface feature, and a dissolvable matrix wherein the plurality of cell adhesion molecules and the plurality of cell capture molecules are modified to attach to the dissolvable matrix. The dissolvable matrix can be attached to a surface. The plurality of cell adhesion molecules can be modified with a plurality of biotin molecules to attach to a plurality of streptavidin molecules on the dissolvable matrix. The plurality of cell capture molecules can be modified with a plurality of biotin molecules to attach to a plurality of streptavidin molecules on the dissolvable matrix. The dissolvable matrix can be alginate an hydrogel. The dissolvable matrix can be dissolved by a chelating agent, enzyme, or combination thereof. The chelating agent can be EDTA, EGTA, or sodium citrate. The plurality of cell adhesion molecules can comprise fibronectin, laminin, collagen, osteopontin, chitosan, chondroitin-sulfate, or hyaluronate. The plurality of cell capture molecules can comprise an antibody, an antigen-specific aptamer, or an antigen-binding antibody fragment. The first cell surface feature can comprise CD44, a variant of CD44, or HABP1. The second cell surface feature can comprise CD44, CD47, MET, EpCAM, CD34, CD38, CD19, Stro1, CD105, CD133, ESA, CD24, ALDH, ALDH1, CD166, SP, CD20, CD117, A2β1, EGFR, HER2, ERCC1, CXCR2, CXCR4, E-Cadherin, Mucin-1, Cytokeratin, PSA, PSMA, STEAP1, RRM1, Androgen Receptor, Estrogen Receptor, progesterone Receptor, IGF1, EML4, Leukocyte Associated Receptor (LAR), or any combination thereof.

Disclosed herein is a method of isolating rare cells or rare cell clusters comprising contacting a biomimetic coating as described herein with a sample containing rare cells at a flow velocity less than 20 mm/s along a coated pathlength, capturing a rare cell on the biomimetic coating; and detecting the rare cell bound by a cell capture molecule wherein a viability of the rare cells is maintained. The coated pathlength can be greater than 20 mm. The rare cells can be maintained at 4° C. The sample can be selected from the group comprising whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total white blood cells, lymph node cells, spleen cells, and tonsil cells. The sample can be treated with an anti-clotting agent. Detecting can comprise microscopy or flow cytometry. The method can comprise contacting the biomimetic coating comprising a captured rare cell with media to maintain the viability of the captured rare cell. The method can comprise analyzing the isolated cells, wherein analysis comprises one or more of image analysis, cell number analysis, cell morphology analysis, polymerase chain reaction (PCR) analysis, sequence analysis, DNA analysis, RNA analysis, gene expression profiling, proteome analysis, metabolome analysis, immunoassays, RNA analysis, gene expression profiling, epigenetic analysis, proteome analysis, metabolome analysis, immunoassays, and nuclear exclusion analysis.

Disclosed herein is a microfluidic device for capturing and maintaining a rare cell or rare cell cluster viable having a capture zone wherein the capture zone comprises a nonporous substrate, a releasable cell adhesion reagent that specifically interacts with a first rare cell surface marker on the rare cell or rare cell cluster wherein the cell adhesion reagent is immobilized on the nonporous substrate, a releasable cell capture reagent that specifically binds a second rare cell surface marker on the rare cell or rare cell cluster wherein the cell capture reagent is immobilized on the nonporous substrate, and a detector for detecting the rare cell or cell cluster bound by the cell capture reagent, wherein the microfluidic device is configured to detect one or more of a rare cell, a rare cell cluster or a bulk tumor cell cluster. The releasable cell adhesion reagent can comprise glycosaminoglycans.

The releasable cell adhesion reagent can comprise fibronectin, laminin, collagen, osteopontin, chitosan, chondroitin sulfate, or hyaluronate. The first rare cell surface marker can comprise CD44, a variant of CD44, or HABP1. The second rare cell surface marker can comprise CD44, CD47, MET, EpCAM, CD34, CD38, CD19, Stro1, CD105, CD133, ESA, CD24, ALDH, ALDH1, CD166, SP, CD20, CD117, A2β1, EGFR, HER2, ERCC1, CXCR2, CXCR4, E-Cadherin, Mucin-1, Cytokeratin, PSA, PSMA, STEAP1, RRM1, Androgen Receptor, Estrogen Receptor, progesterone Receptor, IGF1, EML4, Leukocyte Associated Receptor (LAR), or any combination thereof. The releasable cell capture reagent and the releasable cell adhesion reagent can be bound to a dissolvable matrix. The dissolvable matrix can be an alginate hydrogel. The dissolvable matrix can be dissolvable by a chelating agent, enzyme or combination thereof. The chelating agent can be EDTA, EGTA, or sodium citrate. The microfluidic device can be manufactured using 3D printing technology, photolithography, or a combination thereof.

Disclosed herein is a method of isolating a rare cell, rare cell cluster, bulk tumor cell, or bulk tumor cell cluster comprising introducing a fluid sample into a microfluidic device and causing the rare cell, rare cell cluster, bulk tumor cell, or bulk tumor cell cluster of the fluid sample to traverse a capture zone of the microfluidic device, thereby isolating the rare cell, rare cell cluster, bulk tumor cell, or bulk tumor cell cluster. The capture zone can comprise a nonporous substrate, a releasable cell adhesion reagent that specifically interacts with a first rare cell surface marker on the rare cell or rare cell cluster wherein the cell adhesion reagent is immobilized on the nonporous substrate, a releasable cell capture reagent that specifically binds a second rare cell surface marker on the rare cell or rare cell cluster wherein the cell capture reagent is immobilized on the nonporous substrate, and a detector for detecting the rare cell or cell cluster bound by the cell capture reagent, wherein the microfluidic device is configured to detect one or more of a rare cell, a rare cell cluster or a bulk tumor cell cluster. The method can comprise a flow rate from about 1 mm/s to about 20 mm/s. The sample can be selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total white blood cells, lymph node cells, spleen cells, and tonsil cells. The sample can be treated with an anti-clotting agent. The method can comprise flowing media into the microfluidic device containing isolated rare cells to maintain viability of the isolated rare cells after isolation. The method can comprise maintaining the microfluidic device at a temperature of 4° C. The method can comprise determining a targeted therapy in a subject diagnosed with cancer comprising contacting the biomimetic coating described herein with a sample containing rare cells at a flow velocity less than 20 mm/s along a coated pathlength, capturing a rare cell on the biomimetic coating wherein a viability of the rare cell is maintained, detecting the rare cell bound by a cell capture molecule, removing the rare cell from the biomimetic coating, performing genome sequencing of the rare cell, determining a mutation in the cells, and determining a target therapeutic regime to target the mutation. The method can comprise administering one or more chemotherapeutic agents to the subject. The sample can be selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total while blood cells, lymph node cells, spleen cells, and tonsil cells. Detection can be performed by microscopy or flow cytometry. The cancer can be bladder cancer, bone cancer, brain cancer, breast cancer, cervical cancer, colon cancer, esophageal cancer, gastric cancer, glioma, head and neck cancer, kidney cancer, leukemia, acute myeloid leukemia, multiple myeloma, ovarian cancer, lung cancer, lymphoma, melanoma, mesothelioma, medulloblastoma, hematopoietic cancer, ovarian cancer, pancreatic cancer, prostate cancer, rectal cancer, skin cancer, testicular cancer, tracheal cancer, and vulvar cancer.

Disclosed herein is a method for determining responsiveness of a subject to a therapeutic regime comprising introducing a fluid sample obtained from the subject into a microfluidic device comprising and causing a rare cell cluster, bulk tumor cell, or bulk tumor cell cluster of the fluid sample to traverse a capture zone and isolating and analyzing the rare cell, rare cell cluster, bulk tumor cell, or bulk tumor cell cluster wherein analysis comprises comparing a parameter of the rare cell, rare cell cluster, bulk tumor cell, or bulk tumor cell cluster to a reference parameter, thereby determining the responsiveness of the subject to a therapeutic regime. The capture zone can comprise a nonporous substrate, a releasable cell adhesion reagent that specifically interacts with a first rare cell surface marker on the rare cell or rare cell cluster wherein the cell adhesion reagent is immobilized on the nonporous substrate, a releasable cell capture reagent that specifically binds a second rare cell surface marker on the rare cell or rare cell cluster wherein the cell capture reagent is immobilized on the nonporous substrate, and a detector for detecting the rare cell or cell cluster bound by the cell capture reagent, wherein the microfluidic device is configured to detect one or more of a rare cell, a rare cell cluster or a bulk tumor cell cluster. The sample can be selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total while blood cells, lymph node cells, spleen cells, and tonsil cells. The sample can be treated with an anti-clotting agent. Analyzing can comprise one or more of image analysis, cell number analysis, cell morphology analysis, polymerase chain reaction (PCR) analysis, sequence analysis, DNA analysis, RNA analysis, gene expression profiling, epigenetic analysis, proteome analysis, metabolome analysis, immunoassays, and nuclear exclusion analysis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application contains at least one drawing executed in color. Copies of this patent or patent application with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C show a model of a biomimetic tethering approach on a CSC3 capture zone.

FIGS. 2A-2C shows a calculated particle—boundary interactions within the capture zone of a CSC3 chip at varying channel dimensions, herringbone spacing, and increasing flow rates.

FIG. 3 shows a graph of the pressure gradient across the capture zone of the CSC3.

FIG. 4 shows a PDMS chip plasma bonded to a glass microscope slide with four capture zones and access ports.

FIG. 5 shows a CSC3 capture zone coated with cross-linked alginate derivatized with Cy5 labeled streptavidin.

FIG. 6 shows a freeze frame image of GFP-labeled PC-3 cells flowing on an alginate hydrogel coated CSC3 chip.

FIG. 7A-7B show CSC3 channels with a FITC-labeled streptavidinylated alginate coating (green) with a primary biotinylated anti-human CD38 antibody attached to the streptavidin derivatized alginate hydrogel identified by an anti mouse IgG1 secondary antibody labeled with an Alexa Fluor 594 (orange).

FIG. 8 is a detailed section of a CSC3 channel with immobilized biotinylated and Cy3-labeled hyaluronic acid (green) on the Cy5-streptavidin derivatized alginate coating (red).

FIG. 9 is a section of a CSC3 microfluidic device coated with streptavidinylated alginate, functionalized with biotinylated anti human STEAP1 antibodies and biotinylated hyaluronic acid, with immunocaptured e-GFP labeled Prostate Cancer Cell Spheroids.

FIG. 10 is a section of a CSC3 capture zone coated with biotinylated anti human STEAP1 antibodies and biotinylated hyaluronic acid immobilized on a streptavidin enriched alginate coating showing immunocaptured e-GFP labeled Prostate Cancer Cell Spheroids.

### DETAILED DESCRIPTION OF THE INVENTION

### Overview

Sequestration and immobilization of target cells and clusters from a sample such as whole blood provides unique challenges. Previous approaches for immunocapturing of circulating cells from whole blood have been hampered by the limitations of the low flow rate that such technologies are limited to, 5-20 μL/min. Circulating tumor cells in a blood sample are estimated in the range of <1 to 1 among 1 million white blood cells (WBC). Therefore, a 5-10 mL sample of whole blood may only contain around 20-50 circulating tumor cells. Processing time for a volume of this size using previous approaches is in the range of 4-33 hours for a single sample. Long processing times may negatively impact the rare cell or rare cell cluster viability and therefore should be minimized.

Before the present compositions and methods are described, it is to be understood that this invention is not limited to particular compositions, methods and experimental conditions described, such as compositions, methods, and conditions may vary. It is also to be understood that the terminology use here in for purposes of describing particular embodiments only, and is not intended to be limiting since the scope of the present invention will be limited only in the appended claims.

The present invention allows the optimization of microfluidic parameters of a microfluidic device, such as the CSC3 system described herein, to run efficiently at flow rates around 200 μL/min, with a flow velocity between 1 mm/s and 20 mm/s.

In some embodiments, a 10 mL sample of whole blood can be processed within one hour using the CSC3 system while maintaining the selectivity and efficiency of immunocapturing interactions using the biomimetic coating described herein.

In some embodiments, samples can comprise whole blood, sputum, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total white blood cells, lymph node cells, spleen cells, or tonsil cells. Whole blood can contain preservatives such as citrate, dextrose, phosphate, adenine, etc. Whole blood can contain anti-coagulants such as sodium heparin, EDTA, lithium heparin, sodium citrate, etc.

### Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

A “rare cell” refers to a cell that is typically found in extremely low numbers compared to other cells. For example, 1 cell among 1×10⁹ red blood cells (RBCs). A “rare cell cluster” refers to groups of rare cells that tend to adhere to each other in small groups which may be composed of different cell types. A “rare cell cluster” can be an organoid. Rare cells have been identified using varied nomenclature such as: Cancer Stem Cells (CSCs), Cancer Stem Cell Clusters (CSCCs), Circulating Tumor Cells (CTCs), Circulating Tumor Cell Clusters, Circulating Tumor Clusters, Circulating Tumor Aggregates, Endothelial Progenitor Cells (EPCs), Circulating Tumor Micro-emboli (CTM), and Metastasis Initiating Cells (MICs). Herein, unless otherwise indicated, the term “CSC” includes any, some, or all such terms unless the context otherwise requires.

“Diagnosing” includes determining, monitoring, conformation, subclassification, and prediction of the relevant disease, complication or risk. “determining” relates to becoming aware of a disease, complication, risk, etc. “Monitoring” relates to keeping track of an already diagnosed disease, complication, or risk factor, e.g., to analyze the progression of the disease or the influence of a particular treatment on the progression of disease or complication. “Confirmation” relates to the strengthening or substantiating of a diagnosis already performed using other indicators or markers. “classification” or “subclassification” relates to further defining a diagnosis according to different subclasses of the diagnosed disease, disorder, or condition, e.g., defining according to mild, moderate, or severe forms of the disease or risk. “Prediction” relates to prognosing a disease, disorder, condition, or complication before other symptoms or markers have become evident or have become significantly altered.

A “subject” is a member of any animal species, preferably a mammalian species, optionally a human. Thus, the methods and compositions described herein are applicable to both human and veterinary disease. Further, while a subject is preferably a living organism, the invention described herein may be used in post-mortem analysis as well. Preferred subjects are humans, and most preferably “patients”, which as used herein refers to living humans that are receiving medical care for a disease or condition. This includes persons with no defined illness who are being investigated for signs of pathology. The subject can be an apparently healthy individual, and individual suffering from a disease, or an individual being treated for a disease. A “reference subject” or “reference subjects” is/are an individual or a population that serves as a reference against which to assess another individual or population with respect to one or more parameters.

An “analyte” refers to a substance to be detected, which may be suspected of being present in the sample (i.e. the biological sample). The analyte can be any substance for which there exists a naturally occurring specific binding partner or for which a specific binding partner can be prepared. Thus, an analyte is a substance that can bind to one or more specific binding partners in an assay.

A “binding partner” is a member of a binding pair, i.e., a pair of molecules wherein one of the molecules binds to the second molecule. Binding partners that bind specifically are termed “specific binding partners”. In addition to antigen and antibody binding partners commonly used in immunoassays, other specific binding partners can include biotin and avidin (or streptavidin), carbohydrates and lectins, nucleic acids with complementary nucleotide sequences, effector ligands and receptor molecules, cofactors and enzymes, aptamers and their specific target molecules, etc. Furthermore, specific binding partners can include partner(s) that is/are analog(s) of the original specific binding partner, for example, an analyte-analog. Immunoreactive specific binding partners include antigens, antigen fragments, antibodies and antibody fragments, both monoclonal and polyclonal and complexes thereof, including those formed by recombinant DNA methods.

As used herein, the term “epitope” or “epitopes” or “epitopes of interest” refer to a site(s) on any molecule that is recognized and is capable of binding to a complementary site(s) on its specific binding partner. The epitope-bearing molecule and specific binding partner and part of a specific binding pair. For example, an epitope can be a polypeptide, protein, hapten, carbohydrate antigen (such as, but not limited to, glycolipids, glycoproteins or lipopolysaccharides) or polysaccharide and its specific binding partner. Typically an epitope is contained within a larger molecular framework (e.g., in the context of an antigenic region of a protein, the epitope is the region or fragment of the protein having the structure capable of being bound by an antibody reactive against that epitope) and refers to the precise residues known to contact the specific binding partner. As is known, it is possible for an antigen or antigenic fragment to contain more than one epitope.

As used herein, “specific” or “specificity” in the context of an interaction between members of a specific binding pair (e.g., an antigen and antibody, an aptamer and its specific biomolecular target, etc.) refers to the selective reactivity of the interaction. The phrase “specifically binds to” and analogous terms thereof refer to the ability of one member of a binding pair to specifically bind to (e.g., preferentially react with) the other member of the binding pair and not to bind specifically to other entities. Antibodies, antibody fragments, and other binding pair members that specifically bind to another molecule correlated with cancer can be identified, for example, by diagnostic immunoassays (e.g., radioimmunoassays (“RIA”) and enzyme-linked immunosorbent assay (“ELISAs”), surface plasmon resonance, or other techniques known to those of skill in the art. In one embodiment, the term “specifically binds” or “specifically reactive” indicates that the binding preference (e.g., 10-fold, 100-fold, 1,000-fold, a million-fold, or more over a non-specific target molecule (e.g., ka randomly generated molecule lacking the specifically recognized binding site(s)).

An antigen, antibody, or other analyte “correlated” or “associated” with a disease, particularly cancer refers to an antigen, antibody, or other analyte as the case may be that is positively correlated with the presence or occurrence of cancer generally or a specific type of cancer, as the context requires. In general, an “antigen” is any substance that exhibits specific immunological reactivity with a target antibody. Suitable antigens may include, without limitation, molecules comprising at least one antigenic epitope capable of interacting specifically with the variable region or complementarity determining regions (CDRs) of an antibody or CDR-containing antibody fragment. Antigens typically are naturally occurring or synthetic biological macromolecules such as a protein, peptide, polysaccharide, lipids, or nucleic acids, or complexes containing these or other molecules. As used herein with reference to endogenous cancer (or other disease-associated) antigens (or other analytes correlated with cancer or other disease), the term “elevated level” refers to a level in a sample that is higher than a normal level or range, or is higher than another reference level or range (e.g., earlier or baseline sample).

The term “altered level” refers to a level in a sample that is altered (increased or decreased) over a normal level or range, or over another reference level or range (e.g., earlier or baseline sample). The normal level or range for endogenous cancer antigens is defined in accordance with standard practice. Because the levels of target analyte in some instances will be very low, a so-called altered level or alteration can be considered to have occurred when there is any net change as compared to the normal level or range, or reference level or range that cannot be explained by experimental error or sample variation. Thus, the level measured in a particular sample will be compared with the level or range of levels determined in similar samples of normal tissue. In this context, “normal tissue” is tissue from an individual with no detectable cancer pathology, and a “normal” (sometimes termed “control”) patient (i.e., subject) or population is one that exhibits no detectable pathology. The level of an analyte is said to be “elevated” where the analyte is normally undetectable (e.g., the normal level is zero, or within a range of from about 25 to about 75 percentiles of normal populations), but is detected in a test sample, as well as where the analyte is present in the test sample at a higher than normal level.

A “microarray” or “array” refers to a device consisting of a substrate, typically a solid support having a surface adapted to receive and immobilize a plurality of different protein, peptide, and/or nucleic acid species (i.e., capture or detection reagents) that can be used to, for example, bind to or determine the presence and/or amount of other molecules (i.e., analytes) in biological samples such as blood. “Microarray” or “array” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Ordered arrays include, but are not limited to, those prepared by photolithography, spotting, printing, electrode arrays, “gel pad” arrays, and the like. The size of array can vary from one element to thousands, tens of thousands, or even millions of elements. Depending on the number of array elements required, some array types or methods of preparing the array may be more advantageous, as those skilled in the art are aware. Typically, the oligonucleotides or polynucleotides are single stranded and are covalently attached to the solid phase support, usually by the 5′-end or a 3′-end. The density of non-overlapping regions containing nucleic acids in a microarray is typically greater than 100 per cm², and more preferably, greater than 1000 per cm². As used herein “microarray” or “array” may also refer to a “random microarray” or “random array”, which refer to an array whose spatially discrete regions of oligonucleotides or polynucleotides are not spatially addressed. That is, the identity of the attached oligonucleotides or polynucleotides is not discernable, at least initially, from its location. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways. Likewise, after formation, microbeads, or oligonucleotides thereof, in a random array may be identified in a variety of ways, including by optical labels, such as fluorescent dye ratios or quantum dots, shape, sequence analysis, or the like.

The term “solid phase” refers to any material or substrate that is insoluble or can be made insoluble by a subsequent reaction. A solid phase can be chosen for its intrinsic ability to attract and immobilize a capture or detection reagent. Alternatively, a solid phase can have affixed thereto a linking agent that has the ability to attract and immobilize a capture agent. The linking agent can, for example, include a charged substance that is oppositely charged with respect to the capture agent itself or to a charged substance conjugated to the capture agent. In general, a linking agent can be any binding partner (preferably specific) that is immobilized on (said to be “attached to”) a solid phase and that has the ability to immobilize a desired capture or detection reagent through a binding or other associative reaction. A linking agent enables the indirect binding of a capture reagent to a solid phase material before the performance of an assay or during the performance of an assay. The solid phase can, for example, be plastic, derivatized plastic, magnetic or non-magnetic metal, glass or silicon, including, for example, a test tube, microtiter well, sheet, bead, microparticle, chip, and other configurations known to those of ordinary skill in the art.

The term “nonporous substrate” means a solid support material or matrix on top of which the microfluidic system of the invention is created using a photolithography or other suitable process. The material is typically poly dimethyl siloxane (PDMS) or poly methyl methacrylate (PMMA) or cyclo-poly olefin co-polymer or other suitable materials known in the art.

A “capture zone” refers to a specific area on the microfluidics system that is composed of many separate flow channels based on a herringbone pattern where the channels are coated with a hydrogel matrix. The channels are coated with a hydrogel matrix that have biomolecular binding agents covalently attached with the ability to capture or bind to targeted antigens on the surface of rare cells and biomolecular adhesion agents covalently attached with the ability to specifically interact with target antigens on the surface of rare cells.

A “cell/cluster retention element” refers to a subsystem within a microfluidics flow path that allows cells or cell clusters to be physically retained while allowing smaller biomolecules and buffers to pass through.

A “separation channel” means a separate subsystem within a microfluidics flow path that allows captured cells, once released, to be separated based on a cells unique size, charge, and surface properties, all of which can be manipulated by buffers and/or coatings on the surface of the separation channel.

A “rare cell/cluster detector” refers to a subsystem within a microfluidics separation channel that can “sense” or detect the passage of either single cells or cell clusters as they pass a fixed point within the channel. Detection can be based on either conductivity with no required label or fluorescence emission from, for example, a pre-labeled antibody or aptamer.

As used herein, term “microparticle” refers to a small particle that is recoverable by any suitable process, e.g., magnetic separation or association, ultracentrifugation, etc. Microparticles typically have an average diameter on the order of about 1 micron or less.

A “capture” or “detection” agent or reagent refers to a binding partner that binds to an analyte, preferably specifically. Capture or detection reagents can be attached to or otherwise associated with a solid phase.

The term “labeled detection reagent” refers to a binding partner that binds to an analyte, preferably specifically, and is labeled with a detectable label or becomes labeled with a detectable label during use in an assay. A “detectable label” includes a moiety that is detectable or that can be rendered detectable. With reference to a labeled detection agent, a “direct label” is a detectable label that is attached, by any means, to the detection agent, and an “indirect label” is a detectable label that specifically binds the detection agent. Thus, an indirect label includes a moiety that is the specific binding partner of a moiety of the detection agent. Biotin and avidin are examples of such moieties that can be employed, for example, by contacting a biotinylated antibody with labeled avidin to produce an indirectly labeled antibody.

The term “indicator reagent” refers to any agent that is contacted with a label to produce a detectable signal. Thus, for example, in conventional enzyme labeling, an antibody labeled with an enzyme can be contacted with a substrate (the indicator reagent) to produce a detectable signal, such as a colored reaction product.

An “antibody” refers to a protein consisting of one or more polypeptides substantially encoded by immunoglobulin genes or fragments of immunoglobulin genes. This term encompasses polyclonal antibodies, monoclonal antibodies, nanobodies and antigen-binding fragments thereof, as well as molecules engineered from immunoglobulin gene sequences. Nanobodies are single-domain, heavy chain only antibodies. Nanobodies can be derived from sharks, llamas, or camels. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Antibodies are generally found in bodily fluids, mainly blood.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains, respectively.

Antibodies exist as intact immunoglobulins or as a number of well-characterized fragments produced by digestion with various peptidases. Thus, for example, pepsin digests an antibody below the disulfide linkages in the hinge region to produce F(ab′)2, a dimer of Fab which itself is a light chain joined to VH-CH1 by a disulfide bond. The F(ab′)2 may be reduced under mild conditions to break the disulfide linkage in the hinge region thereby converting the (Fab)2 dimer into a Fab′ monomer. The Fab′ monomer is essentially a Fab with part of the hinge region. While various antibody fragments are defined in terms of the digestion of an intact antibody, one of skill will appreciate that such Fab′ fragments may be synthesized de novo either chemically or by utilizing recombinant DNA methodology. Thus, in the context of the invention, the term “antibody” also includes antigen-binding antibody fragments either produced by the modification of whole antibodies or synthesized de novo using recombinant DNA methodologies. Antibodies include single chain antibodies (antibodies that exist as a single polypeptide chain), single chain Fv antibodies (sFv or scFv), in which a variable heavy and a variable light chain are joined together (directly or through a peptide linker) to form a continuous polypeptide. The single chain Fv antibody is a covalently linked VH-VL heterodimer that may be expressed from a nucleic acid including VH- and VL-encoding sequences either joined directly or joined by a peptide-encoding linker. While the VH and VL are connected to each as a single polypeptide chain, the VH and VL domains associate non-covalently. The scFv antibodies and a number of other structures convert the naturally aggregated, but chemically separated, light and heavy polypeptide chains from an antibody V region into a molecule that folds into a three-dimensional structure substantially similar to the structure of an antigen-binding site are known to those of skill in the art.

An “aptamer” refers to a synthetic oligonucleotide or peptide molecule that binds to a specific target biomolecule, for example, a specific cell surface protein. In addition to their discriminate recognition, aptamers offer advantages over antibodies as they can be engineered completely in a test tube, can be readily produced by chemical synthesis, and possess desirable storage properties. Nucleic acid aptamers are oligonucleotide species that have been engineered through repeated rounds of in vitro selection or, equivalently, SELEX. Both DNA and RNA aptamers show robust binding affinities for various targets. Peptide aptamers are proteins that are designed to bind to specific proteins. They typically consist of a variable peptide loop of about 10-20 amino acids attached at both ends to a protamersein scaffold. This double structural constraint greatly increases the binding affinity of peptide aptamers to levels comparable to an antibody's (nanomolar range). Peptide aptamers can be selected using different systems, including a yeast two-hybrid system as well as from biopanning combinatorial peptide libraries constructed by, for example, phage display.

A “panel” refers to a group of two or more distinct molecular species that have shown to be indicative of or otherwise correlated with a particular disease or health condition. Such “molecular species” may be referred to as “biomarkers”, with the term “biomarker” being understood to mean a biological molecule the presence or absence of which serves as an indicator of a particular biological state, for example, the occurrence (or likelihood of the occurrence) of cancer in a subject. In other words, a biomarker is a characteristic that can be objectively measured and evaluated as an indicator of normal biologic processes, pathogenic processes, or pharmacologic responses to a therapeutic intervention. In the context of the invention an “assay panel” or “array panel” refers to an article, typically a solid phase substrate, having a panel of capture reagents associated therewith (typically by immobilization), wherein at least one of the capture reagents is specifically reactive with an endogenous cancer antigen present on the surface of a CSC. In some embodiments, an assay panel includes 1, 2, 3, 4, 5, 6, 7, 8, 9, 10, 11, 12, 13, 14, 15, 16, 17, 18, 19, 20, or more (e.g., 25, 30, 35, 40, 50, 75, 100, 150, 200, 250, 500, etc., including any integer, or range of integers from 1 to 500) different detection reagents that are proteinaceous cancer-associated antigens, alone or combination with other detection reagents (e.g., nucleic acid-based detection reagents, etc.) correlated with the presence of disease (e.g., cancer) in a subject.

A “biological sample” is a sample of biological material taken from a patient or subject. Biological samples include samples taken from bodily fluids and tissues (e.g., from a biopsy) or tissue preparations (e.g., tissue sections, homogenates, etc.). A “bodily fluid” is any fluid obtained or derived from a subject suitable for use in accordance with the invention. Such fluids include whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total white blood cells, lymph node cells, spleen cells, and tonsil cells.

A “companion diagnostic” is a diagnostic test designed to identify subgroups of patients who may or may not benefit from a particular drug, who may have adverse reactions to the drug, or may require different dosages of the drug.

The term “drug rescue” refers to a drug or drug candidate in the context of the reevaluation of samples and/or data from discontinued clinical trials or pre-clinical development with new or improved evaluation methods.

An “alginate hydrogel” refers to an anionic polysaccharide-based biopolymer. Crosslinking of the gel can be achieved via calcium ions. Alginate hydrogels can be utilized as support structures for tissue engineering, as delivery vehicles for pharmaceuticals, and as model systems for extracellular matrices used in basic biological studies. The addition of chelating reagents which will tie up the calcium ion cross linker will rapidly dissolve an alginate hydrogel.

A “plurality” means more than one.

The term “sample profiling” refers to a representation of information relating to the characteristics of a biological sample, for example, serum, recorded in a quantified way in order to determine patterns or signatures of biomolecules in the particular sample.

As used herein, the term “about” refers to approximately a +/−10% variation from the stated value. It is to be understood that such a variation is always included in any given value provided herein, whether or not it is specifically referred to.

### Coating Overview

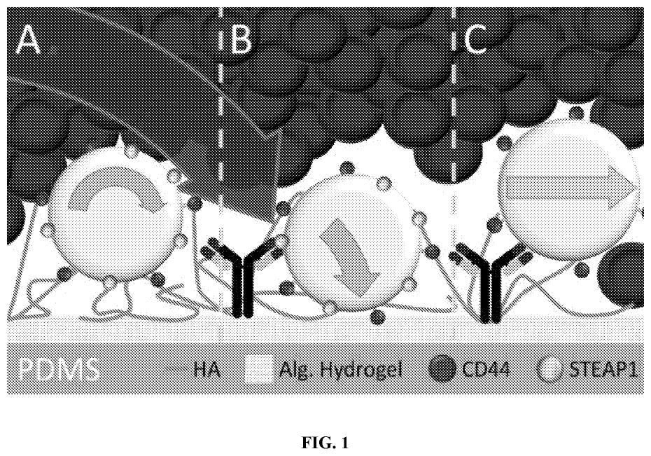

With reference to FIG. 1, the present invention provides a biomimetic coating immobilized on a nonporous substrate. The biomimetic coating can comprise cell capture reagents and cell adhesion reagents immobilized on the nonporous substrate by a dissolvable matrix. In one embodiment, the dissolvable matrix is a hydrogel, such as cellulose hydrogel, glucosaminoglycan (GAG) based hydrogels (such as hyaluronic acid, heparin, heparan sulfate, chondroitin sulfate, dermatan sulfate, or keratin sulfate derived hydrogels), alginate hydrogel, ultrapure sodium alginate, PEGylated alginate hydrogel, or a modified hydrogel to reduce non-specific binding which can be dissolved by a dissolution buffer or agent. Dissolution buffers include those having a chelating agent which acts to dissolve crosslinking of the hydrogel, such as EDTA, EGTA or sodium citrate. Dissolution agents such as enzymes may also be used. In some embodiments the dissolvable matrix is alginate hydrogel derivatized with a covalently bonded streptavidin bio-affinity molecule.

In some embodiments, the cell adhesion element comprises hyaluronic acid. Hyaluronic acid can specifically interact with CD44, a variant of CD44, or HABP1 on the surface of a rare cell. In some embodiments, the hyaluronic acid is functionalized to covalently attach to a hydrogel. In some embodiments, the hyaluronic acid is functionalized with biotin to bind to an alginate hydrogel derivatized with a covalently bonded streptavidin bio-affinity molecule. Any hyaluronic acid molecule with a biotin molecule attached can be attached to an alginate-streptavidin modified hydrogel. The attached hyaluronic acid molecule once immobilized on the alginate hydrogel can specifically interact with a CD44, a variant of CD44 or HABP1 surface antigen of a rare cell or rare cell cluster thus inducing the cell to roll along the hydrogel layer where it can bind to an antibody attached to the alginate hydrogel thus retaining the live cell while other cells are washed away.

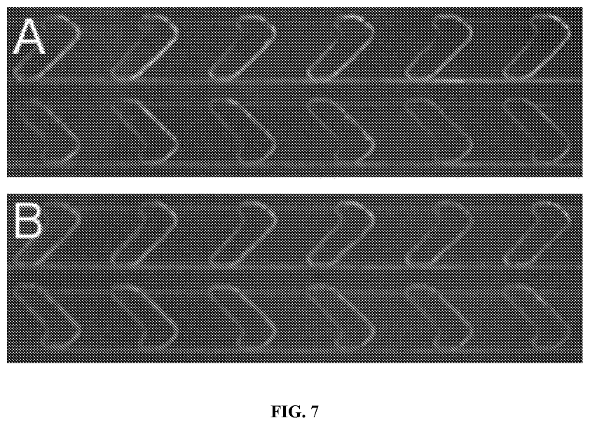

In some embodiments, the cell capture element comprises an antibody, aptamer, or antibody fragment specific to a rare cell surface marker such as CD44, CD47, MET, EpCAM, CD34, CD38, CD90, CD19, Stro-1, CD105, Cd133, ESA, CD24, ALDH, ALDH1, CD166, SP, CD20, Cd117, A2β1, EGFR, HER2, ERCC1, CXCR4, E-Cadherin, Mucin-1, Cytokeratin, PSA, SPMA, RRM1, Androgen Receptor, Estrogen Receptor, Progesterone Receptor, IGF1, EML4, Leukocyte Associated Receptor (LAR), STEAP-1, Myc-1, CD48, CXCR2, ROR-1, ROR-gamma, RTK, TGF-beta, IL-R, TNFR, GF-R, Hedgehog, notch, Wnt, etc. In some embodiments, the cell capture element is functionalized with biotin to bind to an alginate hydrogel derivatized with a covalently bonded streptavidin bio-affinity molecule. In some embodiments, the cell capture element is biotinylated CD38 as can be seen in FIGS. 7A-7B. FIG. 7A is an image of an alginate-streptavidin-FITC coated microfluidic channel with the primary biotinylated anti-human CD38 antibody attached to the streptavidin derivatized alginate hydrogel, 5× objective using the FITC and Cy3 Excitation and Emission filter set. Since the primary antibody has no fluorophore, no signal is generated. FIG. 7B The orange fluorescent signal generated by the secondary antibody, anti-mouse IgG1 with Alexa Fluor 594 is clearly visible indicating the primary antibody is present and coating the channel edges and herringbone extensions.

Any antibody with a biotin molecule attached can be attached to an alginate-streptavidin modified hydrogel. The attached antibody once immobilized on the alginate hydrogel can bind to the surface antigen of a rare cell or rare cell cluster thus retaining the live cell while other cells are washed away.

In some embodiments, the ratio of cell capture element to cell adhesion element attached to the dissolvable layer is between 1:2 and 1:25. In some embodiments, the cell capture element is an antibody. In some embodiments, the cell adhesion element is hyaluronic acid. In some embodiments, the dissolvable layer is alginate. In some embodiments, the molar ratio of antibody to hyaluronic acid attached to the alginate layer is greater than about 2, 3, 4, 5, 6, 7, 8, 9, 10, 11, 12, 13, 14, 15, 16, 17, 18, 19, or 20. In some embodiments, the molar ratio of antibody to hyaluronic acid attached to the alginate layer is less than about 20, 19, 28, 17, 16, 15, 14, 13, 12, 11, 10, 9, 8, 7, 6, 5, 4, 3, or 2.

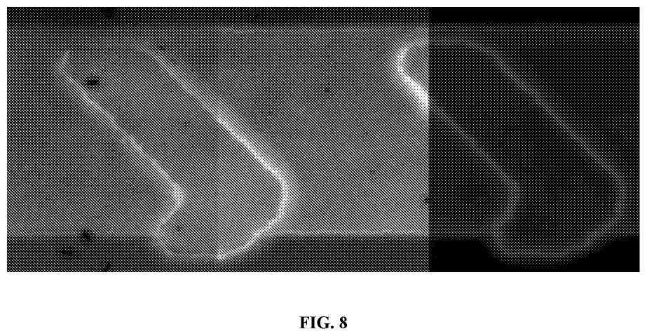

In various embodiments, a channel of a microfluidic device is coated with an alginate-streptavidin modified hydrogel modified with biotinylated antibodies and biotinylated hyaluronic acid molecules, as can be seen in FIG. 8. The hyaluronic acid molecules modified with biotin can be attached to the alginate-streptavidin modified hydrogel with a density greater than about 0.0001 grams per milliliter (g/mL), 0.0002 g/mL, 0.0003 g/mL, 0.0004 g/mL, 0.0005 g/mL, 0.0006 g/mL, 0.0007 g/mL, 0.0008 g/mL, 0.0009 g/mL, 0.0010 g/mL, 0.0011 g/mL, 0.0012 g/mL, 0.0013 g/mL, 0.0014 g/mL, 0.0015 g/mL, 0.0016 g/mL, 0.0017 g/mL, 0.0018 g/mL, 0.0019 g/mL, 0.0020 g/mL, 0.0021 g/mL, 0.0022 g/mL, 0.0023 g/mL, 0.0024 g/mL, 0.0025 g/mL, 0.0026 g/mL, 0.0027 g/mL, 0.0028 g/mL, 0.0029 g/mL, 0.0030 g/mL, 0.0031 g/mL, 0.0032 g/mL, 0.0033 g/mL, 0.0034 g/mL, 0.0035 g/mL, 0.0036 g/mL, 0.0037 g/mL, 0.0038 g/mL, 0.0039 g/mL, 0.0040 g/mL, 0.0041 g/mL, 0.0042 g/mL, 0.0043 g/mL, 0.0044 g/mL, 0.0045 g/mL, 0.0046 g/mL, 0.0047 g/mL, 0.0048 g/mL, 0.0049 g/mL, 0.0050 g/mL, 0.0051 g/mL, 0.0052 g/mL, 0.0053 g/mL, 0.0054 g/mL, 0.0055 g/mL, 0.0056 g/mL, 0.0057 g/mL, 0.0058 g/mL, 0.0059 g/mL, 0.0060 g/mL, 0.0061 g/mL, 0.0062 g/mL, 0.0063 g/mL, 0.0064 g/mL, 0.0065 g/mL, 0.0066 g/mL, 0.0067 g/mL, 0.0068 g/mL, 0.0069 g/mL, 0.0070 g/mL, 0.0071 g/mL, 0.0072 g/mL, 0.0073 g/mL, 0.0074 g/mL, 0.0075 g/mL, 0.0076 g/mL, 0.0077 g/mL, 0.0078 g/mL, 0.0079 g/mL, 0.0080 g/mL, 0.0081 g/mL, 0.0082 g/mL, 0.0083 g/mL, 0.0084 g/mL, 0.0085 g/mL, 0.0086 g/mL, 0.0087 g/mL, 0.0088 g/mL, 0.0089 g/mL, 0.0090 g/mL, 0.0091 g/mL, 0.0092 g/mL, 0.0093 g/mL, 0.0094 g/mL, 0.0095 g/mL, 0.0096 g/mL, 0.0097 g/mL, 0.0098 g/mL, 0.0099 g/mL, 0.0100 g/mL, 0.0110 g/mL, 0.0120 g/mL, 0.0130 g/mL, 0.0140 g/mL, or 0.0150 g/mL. The hyaluronic acid molecules modified with biotin can be attached to the alginate-streptavidin modified hydrogel with a density less than about 0.0150 g/mL, 0.0140 g/mL, 0.0130 g/mL, 0.0120 g/mL, 0.0110 g/mL, 0.0100 g/mL, 0.0099 g/mL, 0.0098 g/mL, 0.0097 g/mL, 0.0096 g/mL, 0.0095 g/mL, 0.0094 g/mL, 0.0093 g/mL, 0.0092 g/mL, 0.0091 g/mL, 0.0090 g/mL, 0.0089 g/mL, 0.0088 g/mL, 0.0087 g/mL, 0.0086 g/mL, 0.0085 g/mL, 0.0084 g/mL, 0.0083 g/mL, 0.0082 g/mL, 0.0081 g/mL, 0.0080 g/mL, 0.0079 g/mL, 0.0078 g/mL, 0.0077 g/mL, 0.0076 g/mL, 0.0075 g/mL, 0.0074 g/mL, 0.0073 g/mL, 0.0072 g/mL, 0.0071 g/mL, 0.0070 g/mL, 0.0069 g/mL, 0.0068 g/mL, 0.0067 g/mL, 0.0066 g/mL, 0.0065 g/mL, 0.0064 g/mL, 0.0063 g/mL, 0.0062 g/mL, 0.0061 g/mL, 0.0060 g/mL, 0.0059 g/mL, 0.0058 g/mL, 0.0057 g/mL, 0.0056 g/mL, 0.0055 g/mL, 0.0054 g/mL, 0.0053 g/mL, 0.0052 g/mL, 0.0051 g/mL, 0.0050 g/mL, 0.0049 g/mL, 0.0048 g/mL, 0.0047 g/mL, 0.0046 g/mL, 0.0045 g/mL, 0.0044 g/mL, 0.0043 g/mL, 0.0042 g/mL, 0.0041 g/mL, 0.0040 g/mL, 0.0039 g/mL, 0.0038 g/mL, 0.0037 g/mL, 0.0036 g/mL, 0.0035 g/mL, 0.0034 g/mL, 0.0033 g/mL, 0.0032 g/mL, 0.0031 g/mL, 0.0030 g/mL, 0.0029 g/mL, 0.0028 g/mL, 0.0027 g/mL, 0.0026 g/mL, 0.0025 g/mL, 0.0024 g/mL, 0.0023 g/mL, 0.0022 g/mL, 0.0021 g/mL, 0.0020 g/mL, 0.0019 g/mL, 0.0018 g/mL, 0.0017 g/mL, 0.0016 g/mL, 0.0015 g/mL, 0.0014 g/mL, 0.0013 g/mL, 0.0012 g/mL, 0.0011 g/mL, 0.0010 g/mL, 0.0009 g/mL, 0.0008 g/mL, 0.0007 g/mL, 0.0006 g/mL, 0.0005 g/mL, 0.0004 g/mL, 0.0003 g/mL, 0.0002 g/mL, or 0.0001 g/mL.

In some embodiments, the hyaluronic acid molecules are between 50 kilodaltons (kDa) and 3000 kDa in length. In some embodiments, the hyaluronic acid molecules are greater than about 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 410 kDa, 420 kDa, 430 kDa, 440 kDa, 450 kDa, 460 kDa, 470 kDa, 480 kDa, 490 kDa, 500 kDa, 510 kDa, 520 kDa, 530 kDa, 540 kDa, 550 kDa, 560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, 710 kDa, 720 kDa, 730 kDa, 740 kDa, 750 kDa, 760 kDa, 770 kDa, 780 kDa, 790 kDa, 800 kDa, 810 kDa, 820 kDa, 830 kDa, 840 kDa, 850 kDa, 860 kDa, 870 kDa, 880 kDa, 890 kDa, 900 kDa, 910 kDa, 920 kDa, 930 kDa, 940 kDa, 950 kDa, 960 kDa, 970 kDa, 980 kDa, 990 kDa, 1000 kDa, 1110 kDa, 1120 kDa, 1130 kDa, 1140 kDa, 1150 kDa, 1160 kDa, 1170 kDa, 1180 kDa, 1190 kDa, 1200 kDa, 1210 kDa, 1220 kDa, 1230 kDa, 1240 kDa, 1250 kDa, 1260 kDa, 1270 kDa, 1280 kDa, 1290 kDa, 1300 kDa, 1310 kDa, 1320 kDa, 1330 kDa, 1340 kDa, 1350 kDa, 1360 kDa, 11370 kDa, 1380 kDa, 1390 kDa, 1400 kDa, 1410 kDa, 1420 kDa, 1430 kDa, 1440 kDa, 1450 kDa, 1460 kDa, 1470 kDa, 1480 kDa, 1490 kDa, 1500 kDa, 1510 kDa, 1520 kDa, 1530 kDa, 1540 kDa, 1550 kDa, 1560 kDa, 1570 kDa, 1580 kDa, 1590 kDa, 1600 kDa, 1610 kDa, 1620 kDa, 1630 kDa, 1640 kDa, 1650 kDa, 1660 kDa, 1670 kDa, 1680 kDa, 1690 kDa, 1700 kDa, 1710 kDa, 1720 kDa, 1730 kDa, 1740 kDa, 1750 kDa, 1760 kDa, 1770 kDa, 1780 kDa, 1790 kDa, 1800 kDa, 1810 kDa, 1820 kDa, 1830 kDa, 1840 kDa, 1850 kDa, 1860 kDa, 1870 kDa, 1880 kDa, 1890 kDa, 1900 kDa, 1910 kDa, 1920 kDa, 1930 kDa, 1940 kDa, 1950 kDa, 1960 kDa, 1970 kDa, 1980 kDa, 1990 kDa, 2000 kDa, 2000 kDa, 2110 kDa, 2120 kDa, 2130 kDa, 2140 kDa, 2150 kDa, 2160 kDa, 2170 kDa, 2180 kDa, 2190 kDa, 2200 kDa, 2210 kDa, 2220 kDa, 2230 kDa, 2240 kDa, 2250 kDa, 2260 kDa, 2270 kDa, 2280 kDa, 2290 kDa, 2300 kDa, 2310 kDa, 2320 kDa, 2330 kDa, 2340 kDa, 2350 kDa, 2360 kDa, 21370 kDa, 2380 kDa, 2390 kDa, 2400 kDa, 2410 kDa, 2420 kDa, 2430 kDa, 2440 kDa, 2450 kDa, 2460 kDa, 2470 kDa, 2480 kDa, 2490 kDa, 2500 kDa, 2510 kDa, 2520 kDa, 2530 kDa, 2540 kDa, 2550 kDa, 2560 kDa, 2570 kDa, 2580 kDa, 2590 kDa, 2600 kDa, 2610 kDa, 2620 kDa, 2630 kDa, 2640 kDa, 2650 kDa, 2660 kDa, 2670 kDa, 2680 kDa, 2690 kDa, 2700 kDa, 2710 kDa, 2720 kDa, 2730 kDa, 2740 kDa, 2750 kDa, 2760 kDa, 2770 kDa, 2780 kDa, 2790 kDa, 2800 kDa, 2810 kDa, 2820 kDa, 2830 kDa, 2840 kDa, 2850 kDa, 2860 kDa, 2870 kDa, 2880 kDa, 2890 kDa, 2900 kDa, 2910 kDa, 2920 kDa, 2930 kDa, 2940 kDa, 2950 kDa, 2960 kDa, 2970 kDa, 2980 kDa, 2990 kDa, or 3000 kDa. In some embodiments, the hyaluronic acid molecules are less than about 3000 kDa, 2990 kDa, 2980 kDa, 2970 kDa, 2960 kDa, 2950 kDa, 2940 kDa, 2930 kDa, 2920 kDa, 2910 kDa, 2900 kDa, 2890 kDa, 2880 kDa, 2870 kDa, 2860 kDa, 2850 kDa, 2840 kDa, 2830 kDa, 2820 kDa, 2810 kDa, 2800 kDa, 2790 kDa, 2780 kDa, 2770 kDa, 2760 kDa, 2750 kDa, 2740 kDa, 2730 kDa, 2720 kDa, 2710 kDa, 2700 kDa, 2690 kDa, 2680 kDa, 2670 kDa, 2660 kDa, 2650 kDa, 2640 kDa, 2630 kDa, 2620 kDa, 2610 kDa, 2600 kDa, 2590 kDa, 2580 kDa, 2570 kDa, 2560 kDa, 2550 kDa, 2540 kDa, 2530 kDa, 2520 kDa, 2510 kDa, 2500 kDa, 2490 kDa, 2480 kDa, 2470 kDa, 2460 kDa, 2450 kDa, 2440 kDa, 2430 kDa, 2420 kDa, 2410 kDa, 2400 kDa, 2390 kDa, 2380 kDa, 2370 kDa, 2360 kDa, 2350 kDa, 2340 kDa, 2330 kDa, 2320 kDa, 2310 kDa, 2300 kDa, 2290 kDa, 2280 kDa, 2270 kDa, 2260 kDa, 2250 kDa, 2240 kDa, 2230 kDa, 2220 kDa, 2210 kDa, 2200 kDa, 2190 kDa, 2180 kDa, 2170 kDa, 2160 kDa, 2150 kDa, 2140 kDa, 2130 kDa, 2120 kDa, 2110 kDa, 2100 kDa, 2090 kDa, 2080 kDa, 2070 kDa, 2060 kDa, 2050 kDa, 2040 kDa, 2030 kDa, 2020 kDa, 2010 kDa, 2000 kDa, 1990 kDa, 1980 kDa, 1970 kDa, 1960 kDa, 1950 kDa, 1940 kDa, 1930 kDa, 1920 kDa, 1910 kDa, 1900 kDa, 1890 kDa, 1880 kDa, 1870 kDa, 1860 kDa, 1850 kDa, 1840 kDa, 1830 kDa, 1820 kDa, 1810 kDa, 1800 kDa, 1790 kDa, 1780 kDa, 1770 kDa, 1760 kDa, 1750 kDa, 1740 kDa, 1730 kDa, 1720 kDa, 1710 kDa, 1700 kDa, 1690 kDa, 1680 kDa, 1670 kDa, 1660 kDa, 1650 kDa, 1640 kDa, 1630 kDa, 1620 kDa, 1610 kDa, 1600 kDa, 1590 kDa, 1580 kDa, 1570 kDa, 1560 kDa, 1550 kDa, 1540 kDa, 1530 kDa, 1520 kDa, 1510 kDa, 1500 kDa, 1490 kDa, 1480 kDa, 1470 kDa, 1460 kDa, 1450 kDa, 1440 kDa, 1430 kDa, 1420 kDa, 1410 kDa, 1400 kDa, 1390 kDa, 1380 kDa, 1370 kDa, 1360 kDa, 1350 kDa, 1340 kDa, 1330 kDa, 1320 kDa, 1310 kDa, 1300 kDa, 1290 kDa, 1280 kDa, 1270 kDa, 1260 kDa, 1250 kDa, 1240 kDa, 1230 kDa, 1220 kDa, 1210 kDa, 1200 kDa, 1190 kDa, 1180 kDa, 1170 kDa, 1160 kDa, 1150 kDa, 1140 kDa, 1130 kDa, 1120 kDa, 1110 kDa, 1100 kDa, 1090 kDa, 1080 kDa, 1070 kDa, 1060 kDa, 1050 kDa, 1040 kDa, 1030 kDa, 1020 kDa, 1010 kDa, 1000 kDa, 990 kDa, 980 kDa, 970 kDa, 960 kDa, 950 kDa, 940 kDa, 930 kDa, 920 kDa, 910 kDa, 900 kDa, 890 kDa, 880 kDa, 870 kDa, 860 kDa, 850 kDa, 840 kDa, 830 kDa, 820 kDa, 810 kDa, 800 kDa, 790 kDa, 780 kDa, 770 kDa, 760 kDa, 750 kDa, 740 kDa, 730 kDa, 720 kDa, 710 kDa, 700 kDa, 690 kDa, 680 kDa, 670 kDa, 660 kDa, 650 kDa, 640 kDa, 630 kDa, 620 kDa, 610 kDa, 600 kDa, 590 kDa, 580 kDa, 570 kDa, 560 kDa, 550 kDa, 540 kDa, 530 kDa, 520 kDa, 510 kDa, 500 kDa, 490 kDa, 480 kDa, 470 kDa, 460 kDa, 450 kDa, 440 kDa, 430 kDa, 420 kDa, 410 kDa, 400 kDa, 390 kDa, 380 kDa, 370 kDa, 360 kDa, 350 kDa, 340 kDa, 330 kDa, 320 kDa, 310 kDa, 300 kDa, 290 kDa, 280 kDa, 270 kDa, 260 kDa, 250 kDa, 240 kDa, 230 kDa, 220 kDa, 210 kDa, 200 kDa, 190 kDa, 180 kDa, 170 kDa, 160 kDa, 150 kDa, 140 kDa, 130 kDa, 120 kDa, 110 kDa, 100 kDa, 90 kDa, 80 kDa, 70 kDa, 60 kDa, or 50 kDa.

### Method of Capture

FIG. 1 shows the process by which the biomimetic coating maintains selectivity and efficiency at the increased flow rate. The process is generally described in three stages: adhesion (FIG. 1A), rolling (FIG. 1B) and capture (FIG. 1C). Adhesion results in the circulating tumor cell or circulating tumor cluster to roll along the biomimetic coating in the direction of flow.

The adhesion stage can be performed through the interaction of a first surface antigen, such as CD44, on the surface of the circulating tumor cell with a cell adhesion element such as hyaluronic acid.

The capture stage can be performed through the interaction of a second surface antigen, such as CD38, on the surface of the circulating tumor cell with a cell capture element such as an anti-CD38 antibody. Table 1 shows a list of rare cells associated with types of cancer that express both CD44 and a second surface antigen which can be targeted by a cell capture element such as an antibody.

TABLE 1

| cancer type | marker patterns for CSCs |
|---|---|
| Acute myelogenic leukemia (AML) | CD34⁺/CD38⁻ |
| | CD90⁺ |
| Acute lymphoblastic leukemia | CD34⁺/CD38⁻/CD19⁺ |
| Bone sarcoma | Stro-1⁺/CD105⁺/CD44⁺ |
| Brain tumor | CD133⁺ |
| Breast cancer | ESA⁺/CD44⁺/CD24^{-/low}/lin^{-a} |
| | CD90^{low}/CD44⁺ |
| | CD44⁺/CD24^{-/low}/ALDH1^{high} |
| Colon Cancer | CD133⁺ |
| | ESA^{high}/CD44⁺/(CD166⁺) |
| | CD133⁺/CD44⁺ |
| | CD133⁺/CD24⁻ |
| Colon cancer (metastatic) | CD133⁺/CD44^{low}/CD24⁺ |
| | CD133⁻/CD44⁺/CD24⁻ |
| Endometrial cancer | CD133⁺ |
| | SP⁺ |
| Gall bladder cancer | CD133⁺/CD44⁺ |
| Gastric cancer | CD44⁺ |
| Liver cancer | CD133⁺/CD44⁺ |
| | CD90⁺ |
| | EpCAM⁺ |
| | CD133⁺ |
| Metastatic melanoma | CD20⁺ |
| Ovarian cancer | CD133⁺/ALDH⁺ |
| | CD44⁺/CD117⁺ |
| Pancreatic cancer | CD44⁺/CD24⁺ ESA⁺ |
| Prostate cancer | CD44⁺/α₂β₁^{hi}/CD133⁺ |
| | CD44⁺/CD24⁻ |
| | SP⁺ |
| Renal cell carcinoma | CD105⁺/(CD133⁻/CD24⁻) |
| Head and neck cancer | CD44⁺ |

In some embodiments, the sample is whole blood. In some embodiments, the method is facilitated by the margination of circulating tumor cells by red blood cells, providing a greater opportunity for circulating tumor cells to interact with the biomimetic coating. Margination can be controlled by the diameter of the channels within the microfluidic device, the configuration of red blood cells in the channel, and the shape of red blood cells in the channel. The configuration of red blood cells in the channel can be altered by features of the channel, such as shapes and structures to induce a chaotic advective flow. The shape of red blood cells in the channel can be altered by the flow rate and pressure within the channel.

Shapes and structures to induce a chaotic advective flow can comprise a protrusion that extends from the microchannel wall or a recess that retreats into the microchannel wall in order to modify the flow in the microchannel. If the area at the top position of the structure to induce a chaotic advective flow is equal to or greater than the area at the bottom position, the structure to induce a chaotic advective flow can be concave, and if the area at the bottom position is larger than the area at the top position structure to induce a chaotic advective flow can be convex. Structures that induce a chaotic advective flow have a depth, width, and length. The structure to induce a chaotic advective flow can have a shape that is substantially circular, rectangular, quadrilateral, rectangular, check mark, chevron, zigzag, etc. The structure to induce a chaotic advective flow can include surface structure. The surface structure can include notches, waves, holes, check marks, scallops, and other forms.

Shapes and structures to induce a chaotic advective flow can include baffle structures that are substantially straight structures, substantially curved structures, substantially triangular structures, substantially square structures. Structures to induce a chaotic advective flow can comprise angled baffles. Structures to induce a chaotic advective flow can be in a herringbone structure. Structures to induce a chaotic advective flow can be in a staggered herringbone pattern. The herringbone pattern can be a high-low herringbone configuration (HLHC). The HLHC can comprise channels that are offset from one another and which fluidly connect with one another in an alternating pattern to produce an increased channel surface area for binding. The cross-section of each channel can be square or rectangular and each channel can be fluidly connected to an adjacent channel. The channels can be in line, not offset, from one another. A herringbone matrix configuration can include a zigzag shape, for example a zigzag shape having equal angles or a zigzag shape having unequal angles, in particular in the shape of a symmetrical or asymmetrical herringbone pattern, or in the shape of a parallel slash mark [/] pattern, in particular an equidistant parallel slash mark pattern.

The structures to induce a chaotic advective flow can be continuously aligned in groups of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100 or more structures.

Shapes and structures to induce a chaotic advective flow can include a plurality of repetitive and similar surface feature structures. Each similar surface feature structure can include at least one angled wall. Each similar surface feature structure can have a depth that is at least 10%, 20%, 30%, 40%, 50%, 60%, 70%, 80%, 90%, or 100% of the gap distance between opposing microchannel surface. A structure to induce a chaotic advective flow can have one or more angular portions that traverse the width direction of at least one wall of the microchannel. A structure to induce chaotic advective flow can have a bent portion rather than a straight line. A structure to induce a chaotic advective flow can have a continuous chevron or zigzag portion. A structure to induce a chaotic advective flow with at least one angle can be aligned with another surface to induce a chaotic advective flow to form a recess or protrusion with a gap between the two structures. For example, the structures to induce chaotic advective flow can be aligned so as to form an angle without touching, the parts can be made discontinuous.

The width of the surface functional structure portion can be orthogonal to the average flow direction of the bulk flow in a preceding channel. A surface functional structure can have an angle of 85°, 75°, 65°, 55°, 50°, 45°, 40°, 35°, 30°, 25°, 20°, 15°, 10°, or 5°, with respect to a plane perpendicular to the average flow direction of the bulk flow in the preceding channel,

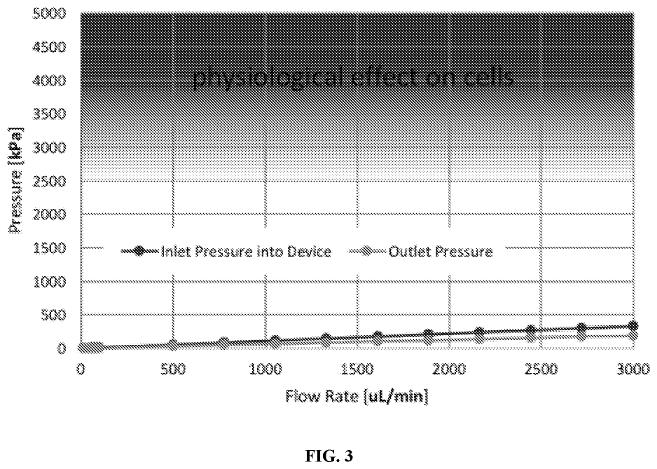

The physiological effect of the pressure induced by higher flow velocities within a CSC3 microfluidic chip was tested, the results of which can be seen in FIG. 3. As can be seen in FIG. 3, the calculated pressure across the microfluidic channels of the CSC3 indicated that a pressure is not reached where physiological changes are induced in the cells. Physiological changes can be caused at a pressure of approximately 2 MPa. With regard to viability, pressures lower than 100 kPa are preferred. FIG. 3 shows that a pressure near 100 kPa was not reached until a flow rate of 1000 μL/min.

In some embodiments, the flow velocity is between 1 millimeter per second (mm/s) and 20 mm/s. In some embodiments, the flow velocity can be greater than about 1 mm/s, 2 mm/s, 3 mm/s, 4 mm/s, 5 mm/s, 6 mm/s, 7 mm/s, 8 mm/s, 9 mm/s, 10 mm/s, 11 mm/s, 12 mm/s, 13 mm/s, 14 mm/s, 15 mm/s, 16 mm/s, 17 mm/s, 18 mm/s, 19 mm/s, or 20 mm/s. In some embodiments, the flow velocity can be less than about 20 mm/s, 19 mm/s, 18 mm/s, 17 mm/s, 16 mm/s, 15 mm/s, 14 mm/s, 13 mm/s, 12 mm/s, 11 mm/s, 10 mm/s, 9 mm/s, 8 mm/s, 7 mm/s, 6 mm/s, 5 mm/s, 4 mm/s, 3 mm/s, 2 mm/s, or 1 mm/s.

In some embodiments, the flow rate is between 15 microliters per minute (μL/min) and 300 μL/min. In some embodiments, the flow rate can be less than about 300 μL/min, 290 μL/min, 280 μL/min, 270 μL/min, 260 μL/min, 250 μL/min, 240 μL/min, 230 μL/min, 220 μL/min, 210 μL/min, 200 μL/min, 190 μL/min, 180 μL/min, 170 μL/min, 160 μL/min, 150 μL/min, 140 μL/min, 130 μL/min, 120 μL/min, 110 μL/min, 100 μL/min, 90 μL/min, 80 μL/min, 70 μL/min, 60 μL/min, 50 μL/min, 40 μL/min, 30 μL/min, 20 μL/min, or 15 μL/min. In some embodiments, the flow rate can be greater than about 15 μL/min, 20 μL/min, 30 μL/min, 40 μL/min, 50 μL/min, 60 μL/min, 70 μL/min, 80 μL/min, 90 μL/min, 100 μL/min, 110 μL/min, 120 μL/min, 130 μL/min, 140 μL/min, 150 μL/min, 160 μL/min, 170 μL/min, 180 μL/min, 190 μL/min, 200 μL/min, 210 μL/min, 220 μL/min, 230 μL/min, 240 μL/min, 250 μL/min, 260 μL/min, 270 μL/min, 280 μL/min, 290 μL/min, or 300 μL/min.

In some embodiments, temperature can be between 0° C. and 40° C. In some embodiments, the temperature is less than about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. In some embodiments, the temperature is greater than about 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 9° C., 8° C., 7° C., 6° C., 5° C., 4° C., 3° C., 2° C., 1° C., or 0° C.

### Use in a Microfluidic Device:

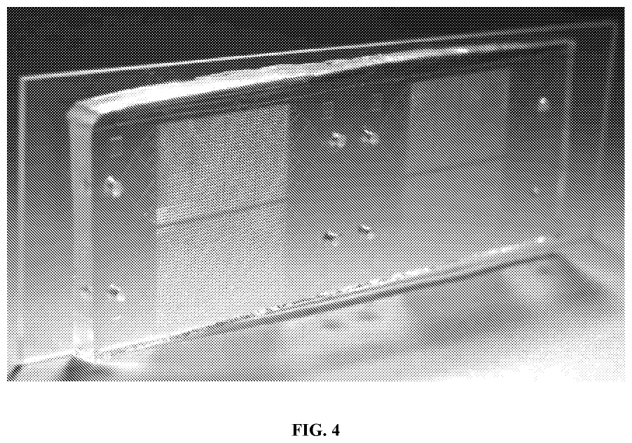

In some embodiments, the coating is used in a microfluidic device for the capture of rare cells. The microfluidic device may be a CSC3 microfluidic device as described in WO 2016/164359, herein incorporated by reference, as can be seen in FIG. 4. A microfluidic device can comprise flow channels coated with the biomimetic coating described herein.

In various embodiments, the width of the flow channels can be from about 5 microns (μm) to about 1000 μm and, for larger width flow channels, can be about 100 μm, at or between about 100 μm and about 150 μm, at or between about 150 μm and 200 μm, at or between about 200 μm and 250 μm, at or between about 250 μm and about 300 μm, at or between about 300 μm and about 350 μm, at or between about 350 μm and about 400 μm, at or between about 400 μm and about 450 μm, at or between about 450 μm and about 500 μm, at or between about 500 μm and about 550 μm, at or between about 550 μm and 600 μm, at or between about 600 μm and about 650 μm, at or between about 650 μm and about 700 μm, at or between about 700 μm and about 750 μm, at or between about 750 μm and 800 μm, at or between about 800 μm and about 850 μm, at or between about 850 μm and about 900 μm, at or between about 900 μm and about 950 μm, at or between 950 μm and 1000 μm. In many applications, a range of flow channel widths from about 75 μm to about 125 μm will be preferred. However, in certain instances, channel widths could exceed 1000 μm. For narrower channels, the widths can be about 5 μm or greater and about 100 μm or smaller. Channel widths can be from about 10 μm to about 75 μm, from about 15 μm to about 50 μm, and from about 20 μm to about 40 μm. In some embodiments the channel width is about 5 μm, 10 μm, 15 μm, 20 μm, 25 μm, 30 μm, 35 μm, 40 μm, 45 μm, 50 μm, 55 μm, 60 μm, 65 μm, 70 μm, or 75 μm. The height can be from about 5 μm to about 100 μm, from about 10 μm to about 75 μm, from about 15 μm to about 50 μm, and from about 20 to about 40 μm. In some embodiments the channel height is about 5 μm, 10 μm, 15 μm, 20 μm, 25 μm, 30 μm, 35 μm, 40 μm, 45 μm, 50 μm, 55 μm, 60 μm, 65 μm, 70 μm, or 75 μm. The cross sectional area can be from about 20 μm² to about 13000 μm², from about 50 to about 10000 μm², from about 200 μm² to about 8000 μm², from about 250 μm² to about 5000 μm², from about 500 μm² to about 3000 μm², and in many embodiments, it is preferred to be from about 1400 μm² to about 1600 μm². In some embodiments the cross sectional area is about 500 μm², 600 μm², 700 μm², 800 μm², 900 μm², 1000 μm², 1100 μm², 1200 μm², 1300 μm², 1400 μm², 1500 μm², 1600 μm², 1700 μm², 1800 μm², 1900 μm², or about 2000 μm². The shape of the cross section of the individual channels of the matrix devices of this invention can be the same or different and can take different shapes such as square, rectangular, other polygonal, circular, elliptical, semicircular, semielliptical, and the like. The cross sectional shapes and areas can vary within the same channel and can be prepared by fabrication techniques described earlier and known in the art. Square or rectangular channel geometries are generally favored.

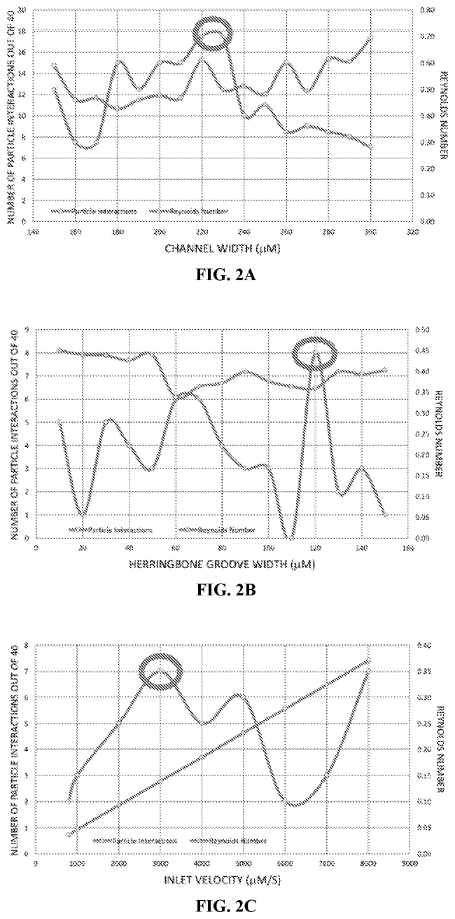

The observed Reynolds numbers (Re) in microfluidic systems often range from 0.2-5, which is substantially below a value of 2300-2900 commonly considered the transition point from laminar to turbulent flow. Laminar flow dramatically reduces the contact time of circulating cells with a biomimetic coating of the non-porous walls of a microfluidic channel. Therefore, microfluidic channels described herein can be designed to introduce chaotic advective flow in order to generate more interactions between rare cells and the channel walls. FIGS. 2A-2C compare the effect of (A) channel width, (C) inlet velocity, and (B) a chaotic advective flow inducing element width (i.e., herringbone groove width) on the number of particle interactions with a wall. As can be seen, the width of the chaotic advective flow inducing element had a significant effect on particle interaction at a lower Reynolds number than (A) or (C).

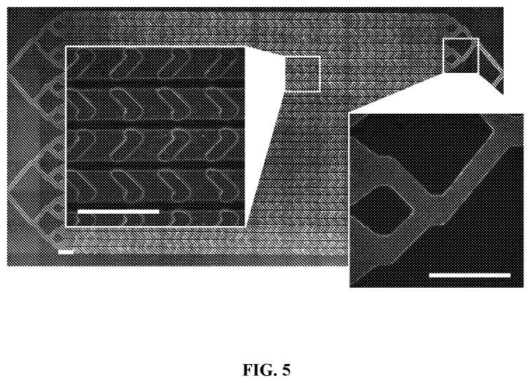

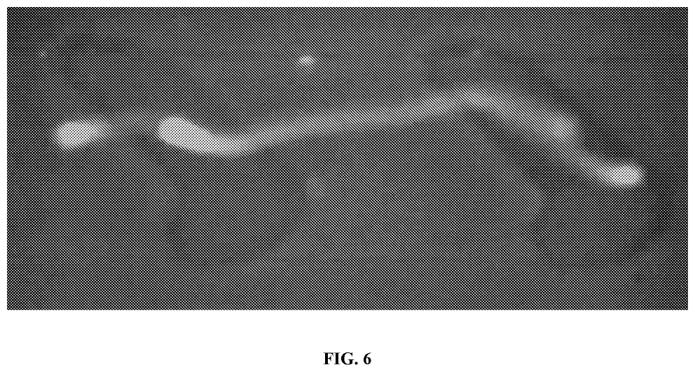

Chaotic advective flow inducing elements can be coated with the biomimetic coating. Channel walls adjacent to the chaotic advective flow inducing element can be coated with the biomimetic coating. Chaotic advective flow inducing elements can include a herringbone (HB) pattern, as can be seen in FIG. 5. FIG. 6 shows a freeze frame illustrating the pronounced lateral movement of GFP-labeled PC3 cells flowing over a chaotic advective flow inducing element coated in alginate hydrogel at a velocity of 5 mm/s. The HB pattern can be an arrangement of checkmark-like contours in an alternating array. The chaotic advective flow inducing elements can be attached to the top of the channel. The chaotic advective flow inducing elements can be attached to the bottom of the channel. The chaotic advective flow inducing elements can be attached to the top and the bottom of the channel. The chaotic advective flow inducing elements can be attached to the sidewall of the channel.

The microfluidic channels described herein can be formed and coated as described in Shaner et al. Design and Production of a Novel Microfluidic Device for the Capture and Isolation of Circulating Tumor Cell Clusters, AIP Advances 9, 065313 (2019), incorporated by reference herein. Microfluidic channels may be generated via a number of methods known in the art. For example, channels may be generated via photolithography, etching, 3D-printing, etc.

Disclosed herein is the use of the biomimetic coating in a microfluidic device to isolate rare cells. The microfluidic device can be used in a method to determine a targeted therapy in a subject diagnosed with cancer comprising outputting a report indicating a subject is positive for cancer, the outputting comprising wherein the presence of the rare cell is diagnostic for the presence of cancer, performing genome sequencing of the rare cell, determining a mutation in the cells, and determining a target therapeutic regime to target the mutation. The method can comprise diagnosing the presence of a cancer in a subject. The method can further comprise administering one or more therapeutic agents to the subject. The method can comprise isolating a panel of rare cells using a panel of cell capture reagents such an antibodies or antibody fragments specific to different target antigens. Detection can be performed by microscopy, microarray, or flow cytometry. Isolated rare cells or rare cell clusters can be analyzed via image analysis. As used herein, image analysis includes any method which allows direct or indirect visualization of rare cells. For example, image analysis may include, but not limited to, microscopic or cytometric detection and visualization of cells bound to a solid substrate, flow cytometry, fluorescent imaging, and the like. In this manner, various parameters of a rare cell may be determined, analyzed and compared to that of a normal cell, including, for example, cellular morphology, such as size and shape.

Therapeutic agents can include chemotherapeutic agents, immunotherapy, growth inhibitory agents, cytotoxic agents, radiation therapy, agents used in radiation therapy, anti-angiogenesis agents, apoptotic agents, anti-tubulin agents, etc, as well as combinations thereof.

The cancer can be bladder cancer, bone cancer, brain cancer, breast cancer, cervical cancer, colon cancer, esophageal cancer, gastric cancer, glioma, head and neck cancer, kidney cancer, leukemia, acute myeloid leukemia, multiple myeloma, ovarian cancer, lung cancer, lymphoma, melanoma, mesothelioma, medulloblastoma, ovarian cancer, hematopoietic cancer, pancreatic cancer, prostate cancer, rectal cancer, skin cancer, testicular cancer, tracheal cancer, and vulvar cancer. The cancer can be lung cancer, pancreas cancer, myeloma, myeloid leukemia, meningioma, glioblastoma, breast cancer, esophageal squamous cell carcinoma, gastric adenocarcinoma, prostate cancer, bladder cancer, ovarian cancer, thyroid cancer, neuroendocrine cancer, colon carcinoma, ovarian cancer, head and neck cancer, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, fibrosarcoma, myxosarcoma, liposarcoma, chondrosarcoma, osteogenic sarcoma, chordoma, angiosarcoma, endotheliosarcoma, Ewing's sarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, endometrial cancer, lung carcinoma, small cell lung carcinoma, bladder carcinoma, epithelial carcinoma, glioblastomas, neuronomas, craniopharingiomas, schwannomas, glioma, astrocytoma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, childhood-null acute lymphoid leukemia (ALL), thymic ALL, B-cell ALL, acute megakaryocytic leukemia, Burkitt's lymphoma, and T cell leukemia, small and large non-small cell lung carcinoma, acute granulocytic leukemia, germ cell tumors, endometrial cancer, gastric cancer, hairy cell leukemia, thyroid cancer, or other known cancer.

Mutations can be determined by next-generation sequencing (NGS), microarrays, fluorescent in situ hybridization (FISH), polymerase chain reaction (PCR), or any combination thereof. Epigenetic biomarkers (such as DNA methylation, such as 5-hydroxymethylated cytosine, 5-methylated cytosine, 5-carboxymethylated cytosine, or 5-formylated cytosine) may be detected by NGS, microarrays, PCR, mass spectrometry (MS), or any combination thereof. Mutations of transcriptomic factors (such as RNA expression levels) may be detected by NGS, microarrays, PCR, or any combination thereof. Proteomic biomarkers (such as a presence of a protein) may be detected by protein arrays, immunohistochemical staining (IHC), or a combination thereof.

Isolated rare cells may be analyzed via nucleic acid sequence analysis, including whole genome sequencing. Completely sequenced genomes may be produced for an individual patient's rare cells or rare cell clusters isolated using a device as described herein and normal (non-cancerous) cells obtained from the patient. Single cell whole genome sequencing can be performed by combining a procedure similar to “Multiple Annealing and Looping Based Amplification Cycles” (MALBAC) with a next generation sequencing (NGS) technology. For example, a whole genome amplification (WGA) method has been reported that allows unbiased uniform amplification of the entire human genome from a single cell (Zong et al. Science Vol. 338, No. 6114, 2012, pp. 1622-1626). The example WGA method is referred to as MALBAC and can be applied to single human cells. MALBAC is a method to pre-amplify the entire genome from an individual cell which sufficient uniformity to accurately sequence greater than 85% of the original cell's genomic DNA. Following the MALBAC process, sufficient quantities of DNA are available for use on any suitable next generation DNA sequencing platform, for example, the Ion Torrent System™ (Thermo Fisher Scientific, Inc.).

The sample can be selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total while blood cells, lymph node cells, spleen cells, and tonsil cells.

Disclosed herein is the use of the biomimetic coating in a microfluidic device to isolate rare cells. The microfluidic device can be used in a method to determine responsiveness of a subject to a therapeutic regime. The method can comprise introducing a fluid sample obtained from the subject into the microfluidic device comprising and causing a rare cell, rare cell cluster, bulk tumor cell, or bulk tumor cell cluster of the fluid sample to be isolated on the biomimetic coating. The method can comprise analyzing the rare cell, rare cell cluster, bulk tumor cell, or bulk tumor cell cluster wherein analysis comprises comparing a parameter of the rare cell, rare cell cluster, bulk tumor cell, or bulk tumor cell cluster to a reference parameter.

The microfluidic device can be used in a method to obtain genetic information from a subject. The method can comprise introducing a fluid sample obtained from the subject into the microfluidic device comprising and causing a rare cell, rare cell cluster, bulk tumor cell, or bulk tumor cell cluster of the fluid sample to be isolated on the biomimetic coating. The method can comprise analyzing the rare cell, rare cell cluster, bulk tumor cell, or bulk tumor cell cluster wherein analysis comprises comparing a parameter of the rare cell, rare cell cluster, bulk tumor cell, or bulk tumor cell cluster to a reference parameter. In this manner is may be desired to determine and identify clinically relevant genetic driver mutations within a cancer stem cell as opposed to non-disease-related genetic differences. As such, a bioinformatics platform(s) which may be utilized in the invention include those from Cypher Genomics, Inc.

The sample volume may be more or less than about 25 μl, 50 μl, 75 μl, 100 μl, 125 μl, 150 μl, 175 μl, 200 μl, 225 μl, 250 μl, 300 μl, 400 μl, 500 μl, 750 μl, 1 ml, 2 ml, 3 ml, 4 ml, 5 ml, 6 ml, 7 ml, 8 ml, 9 ml or greater than about 10 ml, 15 ml, or 20 ml. The sample volume can be between about 1 ml and 2 ml, 2 ml and 3 ml, 3 ml and 4 ml, 4 ml and 5 ml, 5 ml and 6 ml, 6 ml and 7 ml, 7 ml and 8 ml, 8 ml and 9 ml, 9 ml and 10 ml, 10 ml and 11 ml, 11 ml and 12 ml, 12 ml and 13 ml, 13 ml and 14 ml, 14 ml and 15 ml, 15 ml and 16 ml, 16 ml and 17 ml, 17 ml and 18 ml, 18 ml and 19 ml, 19 ml and 20 ml.

The cells of the sample can be treated with an agent that degrades cell clusters to provide for cells that are separated and individual single cells. The cells can be treated prior to (e.g., upstream of) or after (e.g., downstream of) separation within the separation channel (50). An agent that degrades cell clusters includes those that degrade proteins that can be associated with the surface of cells that promote cellular aggregation. The cells can be enzymatically treated to facilitate fibrinolysis. As used herein, fibrinolysis is intended to mean the enzymatic process wherein fibrin and/or products of coagulation, such as fibrin clots and the like are degraded. Degradation by fibrinolysis can be performed by treatment of rare cells with the enzyme plasmin. A variety of natural and synthetic plasmins are well known in the art and may be used with the methods of the present invention so long as the enzyme retains some role in fibrinolysis. In another embodiment, fibrinolysis is produced by enzymatic activation of plasminogen.

In addition to enzymatic degradation cells and proteins aggregated to the surface of rare cells and rare cell clusters, can be treated mechanically, electrically, or chemically. For example, mechanical forces may be used to shear cells and proteins aggregated to their surface. Additionally, treatment with a variety of electrical forces may be utilized such as, but not limited to, electromagnetic, electrostatic, electrochemical, electroradiation, ultrasonic forces, and the like. Electromagnetic radiation can include application of radiation from any region of the electromagnetic spectrum.

In one embodiment, mechanical forces sufficient to breaking up agglomerated rare cell clusters can be generated within the device. This may be performed, for example by generating appropriate physical forces on the cell clusters of a fluid sample flowing through the device by microscale features which may be included along the flow path. Accordingly, rare cells and rare cell clusters may be treated enzymatically, chemically, or the like, after isolation by the device, as well as in the microfluidic device itself.

A population of rare cells isolated by the microfluidic device as disclosed herein. In one aspect, the composition includes unlysed and/or intact cells. In another aspect, the revealed population includes greater than about 1, 2, 3, 4, 5, 7.5, 10, 50, 100, or 200 rare cells per 100 microliters.

Reference parameters can include the number of rare cells, surface antigens of the rare cells, the number of surface antigens on the rare cells, the shape of the rare cells, the composition of clusters of rare cells, etc. Reference parameters can include comparison to a reference sample by one or more of image analysis, cell number analysis, cell morphology analysis, polymerase chain reaction (PCR) analysis, sequence analysis, DNA analysis, RNA analysis, epigenetic analysis, gene expression profiling, proteome analysis, metabolome analysis, immunoassays, and nuclear exclusion analysis.

In some embodiments, the method comprises isolating a panel of rare cells using a panel of cell capture reagents such an antibodies or antibody fragments with a variety of binding partners, i.e., specific to different target antigens. The method can comprise determining the responsiveness of the subject to a therapeutic regime. The method can comprise determining the presence of an altered level of captured cells as compared to a reference sample.

The reference sample can be a from the same subject at a different time point. The reference sample can be from a healthy subject. The reference sample can be from a subject diagnosed with cancer. The reference sample can be from a subject diagnosed with pancreas cancer, myeloma, myeloid leukemia, meningioma, glioblastoma, breast cancer, esophageal squamous cell carcinoma, gastric adenocarcinoma, prostate cancer, thyroid cancer, neuroendocrine cancer, bladder cancer, bone cancer, brain cancer, breast cancer, cervical cancer, colon cancer, esophageal cancer, gastric cancer, glioma, head and neck cancer, kidney cancer, leukemia, acute myeloid leukemia, multiple myeloma, lung cancer, lymphoma, melanoma, mesothelioma, medulloblastoma, ovarian cancer, hematopoietic cancer, pancreatic cancer, prostate cancer, rectal cancer, skin cancer, testicular cancer, tracheal cancer, or vulvar cancer. The reference can be from a subject diagnosed with a disease. The reference sample can be from a subject diagnosed with a tumor, a cancer, a neoplastic or a preneoplastic disease that is characterized by abnormal growth of cells. Non-limiting examples of cancer include lung cancer, pancreas cancer, myeloma, myeloid leukemia, meningioma, glioblastoma, breast cancer, esophageal squamous cell carcinoma, gastric adenocarcinoma, prostate cancer, bladder cancer, ovarian cancer, thyroid cancer, neuroendocrine cancer, colon carcinoma, ovarian cancer, head and neck cancer, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, fibrosarcoma, myxosarcoma, liposarcoma, chondrosarcoma, osteogenic sarcoma, chordoma, angiosarcoma, endotheliosarcoma, Ewing's sarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, endometrial cancer, lung carcinoma, small cell lung carcinoma, bladder carcinoma, epithelial carcinoma, glioblastomas, neuronomas, craniopharingiomas, schwannomas, glioma, astrocytoma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, childhood-null acute lymphoid leukemia (ALL), thymic ALL, B-cell ALL, acute megakaryocytic leukemia, Burkitt's lymphoma, and T cell leukemia, small and large non-small cell lung carcinoma, acute granulocytic leukemia, germ cell tumors, endometrial cancer, gastric cancer, hairy cell leukemia, thyroid cancer, or other known cancer.

In some embodiments, the method comprises analyzing captured rare cells from a series of samples taken from a subject at various time points. The various time points can be before treatment, during exposure to a treatment or after exposure to a treatment. The various time points can be before exposure to a therapeutic, after exposure to a therapeutic, or during exposure to a therapeutic. The time points can include 2 weeks after exposure to a therapeutic or treatment, 1 month, 2 months, 3 months, 4 months, 5 months, 6 months, 7, months, 8 months, 9 months, 10 months, 11 months, 1 year, 2 years, 3 years, 4 years, 5, years, 6 years, 7 years, 8 years, 9 years, or more after exposure to a therapeutic or treatment.

Analysis of the captured rare cell(s) can comprise one or more of image analysis, cell number analysis, cell morphology analysis, polymerase chain reaction (PCR) analysis, sequence analysis, DNA analysis, RNA analysis, epigenetic analysis, gene expression profiling, proteome analysis, metabolome analysis, immunoassays, and nuclear exclusion analysis. Analysis of the captured rare cell(s) can comprise whole genome sequencing, mRNA analysis, next-generation sequencing, etc.

### Example 1: Use in a CSC3 System

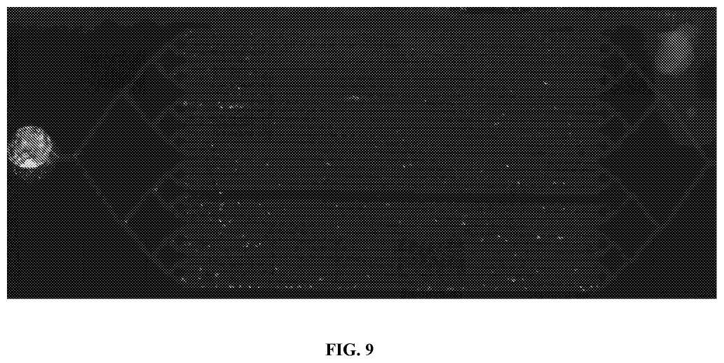

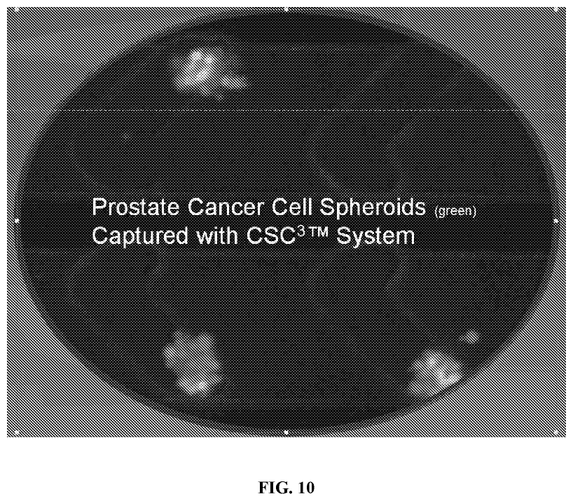

A capture zone of a CSC3 microfluidic device was coated with a streptavidin functionalized alginate hydrogel. The streptavidin functionalized alginate hydrogel was modified with biotinylated hyaluronic acid and biotinylated anti STEAP1 antibody. The coated microfluidic device was flushed overnight at 5 μL/min of 1×TBS with 5% BSA and 0.1% Tween-20. A cell mixture of ˜100,000 MDA-MB-468 cells expressing CD44 and 1,000 FITC labeled PC3 Prostate Cancer Cells expressing CD44 and STEAP1 were injected into the microfluidic device at a flow rate of 25 μL/min. Very few individual MDA breast cancer cells were bound non-specifically. No significant cell lysis was observed. 232 clusters were captured. Out of the 100,000 breast cancer cells that were injected, only about 370, or 0.4% were non-specifically bound. As can be seen in FIGS. 9 and 10, many of the breast MDA-MB-468 breast cancer cells that did stick non-specifically were found to be adhered to the PC3 cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A biomimetic coating to capture rare cells comprising:
a. a plurality of cell adhesion molecules specific to a first cell surface feature;
b. a plurality of cell capture molecules specific to a second cell surface feature; and
c. a dissolvable matrix;
wherein the plurality of cell adhesion molecules and the plurality of cell capture molecules are modified to attach to the dissolvable matrix;
wherein the dissolvable matrix is attached to a surface.

2. The biomimetic coating of claim 1, wherein the plurality of cell adhesion molecules are modified with a plurality of biotin molecules to attach to a plurality of streptavidin molecules on the dissolvable matrix.

3. The biomimetic coating of claim 1, wherein the plurality of cell capture molecules are modified with a plurality of biotin molecules to attach to a plurality of streptavidin molecules on the dissolvable matrix.

4. The biomimetic coating of claim 1, wherein the dissolvable matrix is alginate hydrogel.

5. The biomimetic coating of claim 1, wherein the dissolvable matrix is dissolvable by a chelating agent, enzyme, or combination thereof.

6. The biomimetic coating of claim 5, wherein the chelating agent is EDTA, EGTA, or sodium citrate.

7. The biomimetic coating of claim 1, wherein the plurality of cell adhesion molecules comprises fibronectin, laminin, collagen, osteopontin, chitosan, chondroitin-sulfate, or hyaluronate.

8. The biomimetic coating of claim 1, wherein the plurality of cell capture molecules comprises an antibody, an antigen-specific aptamer, or an antigen-binding antibody fragment.

9. The biomimetic coating of claim 1, wherein the first cell surface feature comprises CD44, a variant of CD44, or HABP1.

10. The biomimetic coating of claim 1, wherein the second cell surface feature comprises CD44, CD47, MET, EpCAM, CD34, CD38, CD19, Stro1, CD105, CD133, ESA, CD24, ALDH, ALDH1, CD166, SP, CD20, CD117, A2β1, EGFR, HER2, ERCC1, CXCR2, CXCR4, E-Cadherin, Mucin-1, Cytokeratin, PSA, PSMA, STEAP1, RRM1, Androgen Receptor, Estrogen Receptor, progesterone Receptor, IGF1, EML4, Leukocyte Associated Receptor (LAR), or any combination thereof.

11. A method of isolating rare cells comprising:
a. contacting the biomimetic coating of claim 1 with a sample containing rare cells at a flow velocity less than 20 mm/s along a coated pathlength;
b. capturing a rare cell on the biomimetic coating; and
c. detecting the rare cell bound by a cell capture molecule;
wherein a viability of the rare cells is maintained.

12. The method of claim 11, wherein the coated pathlength is greater than 20 mm.

13. The method of claim 11, wherein the rare cells are maintained at 4° C.

14. The method of claim 11, wherein the sample is selected from the group comprising whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total white blood cells, lymph node cells, spleen cells, and tonsil cells.

15. The method of claim 14, wherein the sample is treated with an anti-clotting agent.

16. The method of claim 11, wherein detecting comprises microscopy or flow cytometry.

17. The method of claim 11, further comprising contacting the biomimetic coating, comprising a captured rare cell, with media to maintain the viability of the captured rare cell.

18. The method of claim 11, further comprising analyzing the isolated cells, wherein analysis comprises one or more of image analysis, cell number analysis, cell morphology analysis, polymerase chain reaction (PCR) analysis, sequence analysis, DNA analysis, RNA analysis, gene expression profiling, proteome analysis, metabolome analysis, immunoassays, RNA analysis, gene expression profiling, epigenetic analysis, proteome analysis, metabolome analysis, immunoassays, and nuclear exclusion analysis.

19. A microfluidic device for capturing and maintaining a rare cell or rare cell cluster viable having a capture zone wherein the capture zone comprises:
a) a nonporous substrate;
b) a releasable cell adhesion reagent that specifically interacts with a first rare cell surface marker on the rare cell or rare cell cluster wherein the cell adhesion reagent is immobilized on the nonporous substrate;
c) a releasable cell capture reagent that specifically binds a second rare cell surface marker on the rare cell or rare cell cluster wherein the cell capture reagent is immobilized on the nonporous substrate; and
d) a detector for detecting the rare cell or cell cluster bound by the cell capture reagent, wherein the microfluidic device is configured to detect one or more of a rare cell, a rare cell cluster or a bulk tumor cell cluster.

20. The microfluidic device of claim 19, wherein the releasable cell adhesion reagent comprises glycosaminoglycans.

21. The microfluidic device of claim 19, wherein the releasable cell adhesion reagent comprises fibronectin, laminin, collagen, osteopontin, chitosan, chondroitin sulfate, or hyaluronate.

22. The microfluidic device of claim 19, wherein the first rare cell surface marker comprises CD44, a variant of CD44, or HABP1.

23. The microfluidic device of claim 19, the second rare cell surface marker comprises CD44, CD47, MET, EpCAM, CD34, CD38, CD19, Stro1, CD105, CD133, ESA, CD24, ALDH, ALDH1, CD166, SP, CD20, CD117, A2β1, EGFR, HER2, ERCC1, CXCR2, CXCR4, E-Cadherin, Mucin-1, Cytokeratin, PSA, PSMA, STEAP1, RRM1, Androgen Receptor, Estrogen Receptor, progesterone Receptor, IGF1, EML4, Leukocyte Associated Receptor (LAR), or any combination thereof.

24. The microfluidic device of claim 19, wherein the releasable cell capture reagent and the releasable cell adhesion reagent are bound to a dissolvable matrix.

25. The microfluidic device of claim 24, wherein the dissolvable matrix is an alginate hydrogel.

26. The microfluidic device of claim 24, wherein the dissolvable matrix is dissolvable by a chelating agent, enzyme or combination thereof.

27. The microfluidic device of claim 26, wherein the chelating agent is EDTA, EGTA, or sodium citrate.

28. The microfluidic device of claim 19, wherein the microfluidic device is manufactured using 3D printing technology, photolithography, or a combination thereof.

29. A method of isolating a rare cell, rare cell cluster, bulk tumor cell, or bulk tumor cell cluster comprising introducing a fluid sample into a microfluidic device and causing the rare cell, rare cell cluster, bulk tumor cell, or bulk tumor cell cluster of the fluid sample to traverse a capture zone of the microfluidic device, thereby isolating the rare cell, rare cell cluster, bulk tumor cell, or bulk tumor cell cluster; wherein the capture zone comprises
a. a nonporous substrate;
b. a releasable cell adhesion reagent that specifically interacts with a first rare cell surface marker on the rare cell or rare cell cluster wherein the cell adhesion reagent is immobilized on the nonporous substrate;
c. a releasable cell capture reagent that specifically binds a second rare cell surface marker on the rare cell or rare cell cluster wherein the cell capture reagent is immobilized on the nonporous substrate; and
d. a detector for detecting the rare cell or cell cluster bound by the cell capture reagent, wherein the microfluidic device is configured to detect one or more of a rare cell, a rare cell cluster or a bulk tumor cell cluster.

30. The method of claim 28, comprising a flow rate from about 1 mm/s to about 20 mm/s.

31. The method of claim 28, wherein the sample is selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total white blood cells, lymph node cells, spleen cells, and tonsil cells.

32. The method of claim 28, wherein the sample is treated with an anti-clotting agent.

33. The method of claim 28, further comprising flowing media into the microfluidic device containing isolated rare cells to maintain viability of the isolated rare cells after isolation.

34. The method of claim 31, wherein the method comprises maintaining the microfluidic device at a temperature of 4° C.

35. A method of determining a targeted therapy in a subject diagnosed with cancer comprising:
a. contacting the biomimetic coating of claim 1 with a sample containing rare cells at a flow velocity less than 20 mm/s along a coated pathlength;
b. capturing a rare cell on the biomimetic coating wherein a viability of the rare cell is maintained;
c. detecting the rare cell bound by a cell capture molecule;
d. removing the rare cell from the biomimetic coating;
e. performing genome sequencing of the rare cell;
f. determining a mutation in the cells;
g. determining a target therapeutic regime to target the mutation.

36. The method of claim 35, further comprising administering one or more chemotherapeutic agents to the subject.

37. The method of claim 35, wherein the sample is selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total while blood cells, lymph node cells, spleen cells, and tonsil cells.

38. The method of claim 35, wherein detecting is performed by microscopy or flow cytometry.

39. The method of claim 35, wherein the cancer is bladder cancer, bone cancer, brain cancer, breast cancer, cervical cancer, colon cancer, esophageal cancer, gastric cancer, glioma, head and neck cancer, kidney cancer, leukemia, acute myeloid leukemia, multiple myeloma, ovarian cancer, lung cancer, lymphoma, melanoma, mesothelioma, medulloblastoma, hematopoietic cancer, ovarian cancer, pancreatic cancer, prostate cancer, rectal cancer, skin cancer, testicular cancer, tracheal cancer, and vulvar cancer.

40. A method for determining responsiveness of a subject to a therapeutic regime comprising:
a) introducing a fluid sample obtained from the subject into a microfluidic device comprising and causing a rare cell cluster, bulk tumor cell, or bulk tumor cell cluster of the fluid sample to traverse a capture zone, wherein the capture zone comprises:
i. a nonporous substrate;
ii. a releasable cell adhesion reagent that specifically interacts with a first rare cell surface marker on the rare cell or rare cell cluster wherein the cell adhesion reagent is immobilized on the nonporous substrate;
iii. a releasable cell capture reagent that specifically binds a second rare cell surface marker on the rare cell or rare cell cluster wherein the cell capture reagent is immobilized on the nonporous substrate;
iv. a detector for detecting the rare cell or cell cluster bound by the cell capture reagent, wherein the microfluidic device is configured to detect one or more of a rare cell, a rare cell cluster or a bulk tumor cell cluster; and
b) isolating and analyzing the rare cell, rare cell cluster, bulk tumor cell, or bulk tumor cell cluster wherein analysis comprises comparing a parameter of the rare cell, rare cell cluster, bulk tumor cell, or bulk tumor cell cluster to a reference parameter, thereby determining the responsiveness of the subject to a therapeutic regime.

41. The method of claim 40, wherein the sample is selected from whole blood, blood fractions such as serum and plasma, urine, sweat, lymph, feces, ascites, seminal fluid, sputum, nipple aspirate, post-operative seroma, wound drainage fluid, saliva, synovial fluid, ascites fluid, bone marrow aspirate, cerebrospinal fluid, nasal secretions, amniotic fluid, bronchoalveolar lavage fluid, pleural effusion, peripheral blood mononuclear cells, total while blood cells, lymph node cells, spleen cells, and tonsil cells.

42. The method of claim 40, wherein the sample is treated with an anti-clotting agent

43. The method of claim 40, wherein analyzing comprises one or more of image analysis, cell number analysis, cell morphology analysis, polymerase chain reaction (PCR) analysis, sequence analysis, DNA analysis, RNA analysis, gene expression profiling, epigenetic analysis, proteome analysis, metabolome analysis, immunoassays, and nuclear exclusion analysis.
